(19) United States
(12) Patent Application Publication
(10) Pub. No.: US 2025/0089598 A1
(43) Pub. Date: Mar. 20, 2025

(54) SYSTEM AND METHOD FOR DETERMINING COMPACTION LAYER DEPTH DURING AGRICULTURAL MACHINE OPERATION

(71) Applicant: CNH Industrial America LLC, New Holland, PA (US)

(72) Inventors: Brittany Schroeder, Bunker Hill, IN (US); Devon Olson, Barnesville, MN (US)

(21) Appl. No.: 18/468,926

(22) Filed: Sep. 18, 2023

(51) Int. Cl. A01B 79/00; A01B 37/00; A01B 63/00; G01B 11/30; G01N 33/24

(52) U.S. Cl. CPC A01B 79/005; A01B 37/00; A01B 63/008; G01B 11/30; G01N 33/24; G01N 33/245

(57) ABSTRACT

A system for determining compaction layer depth during agricultural machine includes a transceiver-based sensor configured to generate surface profile data indicative of a surface profile of a portion of a field across which the agricultural machine is traveling. Furthermore, the system includes a computing system communicatively coupled to the transceiver-based sensor. As such, the computing system is configured to identify a wheel depression within the portion of the field within the surface profile data generated by the transceiver-based sensor. Additionally, the computing system is configured to determine a depth of the identified wheel depression and determine a depth of a compaction layer beneath the identified wheel depression based on the determined depth of the identified wheel depression.

### FIELD OF THE INVENTION

The present disclosure generally relates to agricultural machines and, more particularly, to systems and methods for determining compaction layer depth during the operation of an agricultural machine.

### BACKGROUND OF THE INVENTION

It is well known that, to attain the best agricultural performance from a piece of land, a farmer must cultivate the soil, typically through a tillage operation. Common tillage operations include plowing, harrowing, and sub-soiling. Modern farmers perform these tillage operations by pulling a tillage implement behind an agricultural work vehicle, such as a tractor. Depending on the crop selection and the soil conditions, a farmer may need to perform several tillage operations at different times over a crop cycle to properly cultivate the land to suit the crop choice.

When performing certain tillage operations, it is generally desirable to break up any layers of subsurface soil that have been compacted (e.g., due to vehicle traffic, ponding, and/or the like). As such, during such tillage operations, shanks or other ground-penetrating tools supported on the tillage implement are pulled through the soil to fracture the compaction layer. However, the depth of the compaction layer may vary throughout the field. In this respect, systems have been developed that allow compaction layers to be detected and the penetration depths of the shanks or other tools to be adjusted accordingly. While such systems work well, further improvements are needed.

Accordingly, an improved system and method for determining compaction layer depth during agricultural machine operation would be welcomed in the technology.

### SUMMARY OF THE INVENTION

Aspects and advantages of the technology will be set forth in part in the following description, or may be obvious from the description, or may be learned through practice of the technology.

In one aspect, the present subject matter is directed to an agricultural machine including a frame and a plurality of wheels coupled to the frame. Furthermore, the agricultural machine includes a transceiver-based sensor supported on the frame, with the transceiver-based sensor configured to generate surface profile data indicative of a surface profile of a portion of a field across which the agricultural machine is traveling. Additionally, the agricultural machine includes a computing system communicatively coupled to the transceiver-based sensor. In this respect, the computing system is configured to identify a wheel depression within the portion of the field based on the surface profile data generated by the transceiver-based sensor. Moreover, the computing system is configured to determine a depth of the identified wheel depression and determine a depth of a compaction layer beneath the identified wheel depression based on the determined depth of the identified wheel depression. In addition, the computing system is configured to control an operating parameter of the agricultural machine based on the determined depth of the compaction layer.

In another aspect, the present subject matter is directed to a system for determining compaction layer depth during agricultural machine operation. The system includes a transceiver-based sensor configured to generate surface profile data indicative of a surface profile of a portion of a field across which the agricultural machine is traveling. Furthermore, the system includes a computing system communicatively coupled to the transceiver-based sensor. As such, the computing system is configured to identify a wheel depression within the portion of the field based on the surface profile data generated by the transceiver-based sensor. Additionally, the computing system is configured to determine a depth of the identified wheel depression and determine a depth of a compaction layer beneath the identified wheel depression based on the determined depth of the identified wheel depression.

In a further aspect, the present subject matter is directed to a method for determining compaction layer depth during agricultural machine operation. The method includes receiving, with a computing system, surface profile data indicative of a surface profile of a portion of a field across which an agricultural machine is traveling. Moreover, the method includes identifying, with the computing system, a wheel depression within the portion of the field based on the received surface profile data. In addition, the method includes determining, with the computing system, a depth of the identified wheel depression. Furthermore, the method includes determining, with the computing system, a depth of a compaction layer beneath the identified wheel depression based on the determined depth of the identified wheel depression. Additionally, the method includes controlling, with the computing system, an operating parameter of the agricultural machine based on the determined depth of the compaction layer.

These and other features, aspects and advantages of the present technology will become better understood with reference to the following description and appended claims. The accompanying drawings, which are incorporated in and constitute a part of this specification, illustrate embodiments of the technology and, together with the description, serve to explain the principles of the technology.

### BRIEF DESCRIPTION OF THE DRAWINGS

A full and enabling disclosure of the present technology, including the best mode thereof, directed to one of ordinary skill in the art, is set forth in the specification, which makes reference to the appended figures, in which:

FIG. 1 illustrates a perspective view of one embodiment of an agricultural machine in accordance with aspects of the present subject matter;

FIG. 2 illustrates partial perspective view of the agricultural machine shown in FIG. 1, particularly illustrating various ground-engaging tools of the machine;

FIG. 3 illustrates a schematic view of one embodiment of a system for determining compaction layer depth during agricultural machine operation in accordance with aspects of the present subject matter;

FIG. 4 illustrates a flow diagram providing one embodiment of control logic for determining compaction layer depth during agricultural machine operation in accordance with aspects of the present subject matter; and

FIG. 5 illustrates a flow diagram of one embodiment of a method for determining compaction layer depth during agricultural machine operation in accordance with aspects of the present subject matter.

Repeat use of reference characters in the present specification and drawings is intended to represent the same or analogous features or elements of the present technology.

### DETAILED DESCRIPTION OF THE DRAWINGS

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield still a further embodiment. Thus, it is intended that the present invention covers such modifications and variations as come within the scope of the appended claims and their equivalents.

In general, the present subject matter is directed to a system and a method for determining compaction layer depth during agricultural machine operation. Specifically, in several embodiments, the system includes one or more transceiver-based sensors configured to generate surface profile data indicative of the surface profile of a portion of a field across which the agricultural machine is traveling. For example, in one embodiment, the transceiver-based sensor(s) may be configured as a LiDAR sensor(s). Moreover, in some embodiments, the transceiver-based sensor(s) may be mounted on or otherwise supported on an agricultural machine (e.g., an agricultural implement and/or an associated work vehicle) such that the transceiver-based sensor(s) have a field(s) of view directed forward of the machine.

Furthermore, a computing system of the disclosed system may be configured to determine the depths of one or more compaction layers using the surface profile data generated by the transceiver-based sensor(s). Specifically, in several embodiments, the computing system is configured to identify one or more wheel depressions or wheel tracks within the portion of the field based on the surface profile data. For example, in one embodiment, the computing system may identify the wheel depression(s) based on shape. Additionally, the computing system is configured to determine the depth(s) of the identified wheel depression(s). Moreover, the computing system is configured to determine the depth(s) of the compaction layer(s) beneath the identified wheel depression(s) based on the determined depth(s) of the identified wheel depression(s). In addition, in some embodiments, the computing system may use the soil texture and/or soil moisture content in addition to the determined depth(s) of the identified wheel depression(s) to identify the depth(s) of the compaction layer(s). Thereafter, the computing system may be configured to control one or more operating parameters of the agricultural machine (e.g., the penetration depth(s) of its ground-engaging tools) based on the determined depth(s) of the compaction layer(s).

The disclosed system and method improve the operation of the agricultural machine. More specifically, the wheels of agricultural machines (e.g., tractors, implements, etc.) and the other vehicles that traverse a field (e.g., grain carts, trucks, etc.) may create subsurface layers of compacted soil known as compaction layers within the field. As such, the weight of the equipment affects the depths of such compaction layers. However, the weight of a given piece of equipment may vary during the operation, such as due to the dispensing of a stored product (e.g., fertilizer, pesticide, etc.) across the field, fuel consumption, field material accumulation thereon, and/or the like. In this respect, as described above, the disclosed system and method determine the depths of the compaction layers beneath the wheel tracks based on the depths of the wheel depressions. Thus, the disclosed system and method provide accurate indications of compaction layer depth beneath wheel depressions in a field even as the weight of the equipment creating such compaction layers varies.

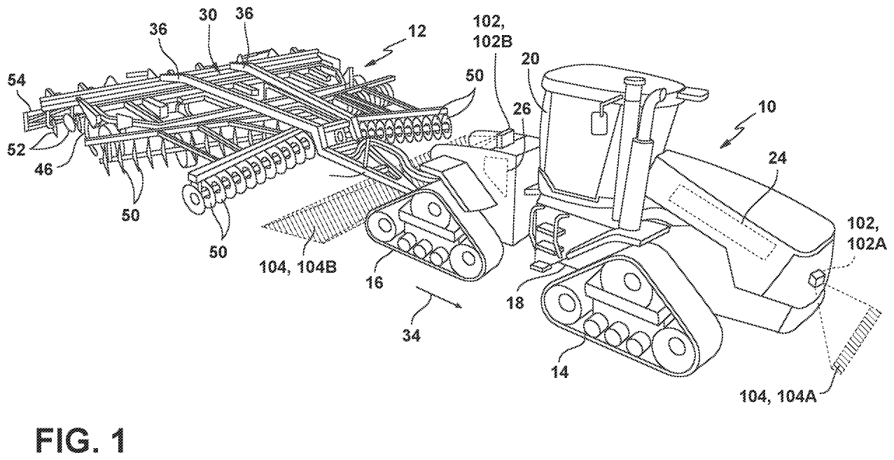

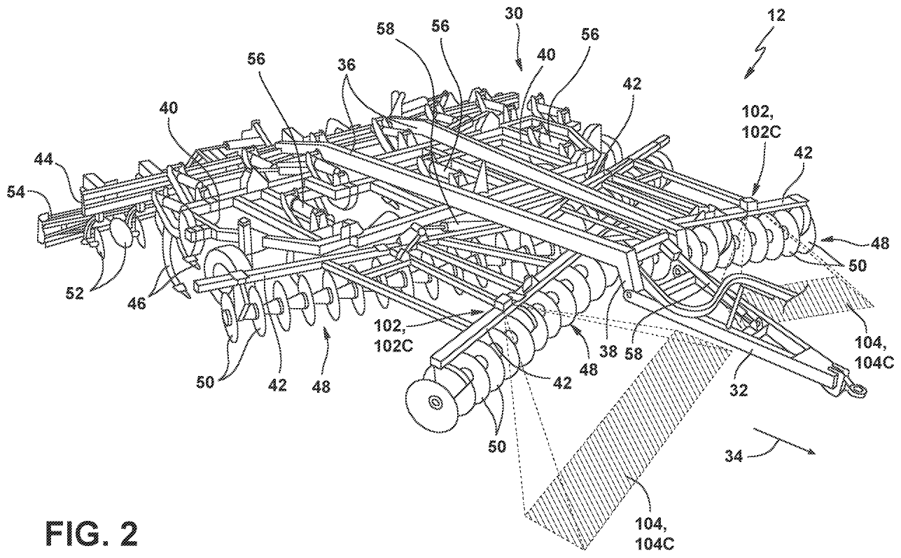

Referring now to drawings, FIGS. 1 and 2 illustrate perspective views of one embodiment of an agricultural machine 10 in accordance with aspects of the present subject matter. Specifically, in the several embodiments, the agricultural machine 10 includes a work vehicle 11 and an associated agricultural implement 12. As shown, the work vehicle 11 may be configured as an agricultural tractor and the agricultural implement 12 may be configured as a tillage implement, such as the illustrated disk ripper. However, in other embodiments, the work vehicle 11 may be configured as any other suitable type of vehicle, such as an agricultural harvester, a self-propelled sprayer, and/or the like. Similarly, in alternative embodiments, the agricultural implement 12 may be configured as any other suitable type of implement. such as a cultivator or other type of tillage implement, a seed-planting implement, a side dresser or another fertilizer dispensing implement, a strip tiller, and/or the like. Furthermore, the agricultural machine 10 may correspond to any suitable powered and/or unpowered agricultural machine (including suitable vehicles and/or equipment, such as only an agricultural vehicle or only an implement). Additionally, the agricultural machine 10 may include only a single machine (e.g., a tractor) or more than two machines.

As particularly shown in FIG. 1, the work vehicle 11 includes a pair of front track assemblies 14, a pair of rear track assemblies 16, and a frame or chassis 18 coupled to and supported by the track assemblies 14, 16. An operator's cab 20 may be supported by a portion of the chassis 18 and may house various input devices for permitting an operator to control the operation of one or more components of the work vehicle 11 and/or one or more components of the agricultural implement 12. Additionally, the work vehicle 11 may include an engine 24 and a transmission 26 mounted on the chassis 18. The transmission 26 may be operably coupled to the engine 24 and may provide variably adjusted gear ratios for transferring engine power to the track assemblies 14, 16 via a drive axle assembly (not shown) (or via axles if multiple drive axles are employed). Furthermore, the work vehicle 11 can have any other suitable traction device(s), such as wheels or tires, and/or any other suitable transmission/engine configuration.

Moreover, as shown in FIGS. 1 and 2, the agricultural implement 12 includes a carriage frame assembly 30 configured to be towed by the work vehicle 11 via a pull hitch or tow bar 32 in a direction of travel of the vehicle (e.g., as indicated by arrow 34). In this respect, the carriage frame assembly 30 may be configured to support a plurality of ground-engaging tools, such as a plurality of shanks, disk blades, leveling blades, basket assemblies, and/or the like. As such, the various ground-engaging tools may be configured to perform an agricultural operation (e.g., a tillage operation) on the field across which the agricultural implement 12 is being towed.

As particularly shown in FIG. 2, the carriage frame assembly 30 may include aft extending carrier frame members 36 coupled to the tow bar 32. In addition, reinforcing gusset plates 38 may be used to strengthen the connection between the tow bar 32 and the carrier frame members 36. In several embodiments, the carriage frame assembly 30 may support a central frame 40, a forward frame 42 positioned forward of the central frame 40 relative to the direction of travel 34 of the work vehicle 11, and an aft frame 44 positioned aft of the central frame 40 relative to the direction of travel 34 of the work vehicle 11. As shown in FIG. 2, in one embodiment, the central frame 40 may correspond to a shank frame configured to support a plurality of ground-engaging shanks 46. In such an embodiment, the shanks 46 may be configured to till the soil as the agricultural implement 12 is towed across the field. However, in other embodiments, the central frame 40 may be configured to support any other suitable ground-engaging tools.

Additionally, as shown in FIG. 2, in one embodiment, the forward frame 42 may correspond to a disk frame configured to support various gangs or sets 48 of disk blades 50. In such an embodiment, each disk blade 50 may, for example, include both a concave side (not shown) and a convex side (not shown). In addition, the various gangs 48 of disk blades 50 may be oriented at an angle relative to the travel direction 34 of the work vehicle 11 to promote more effective tilling of the soil. However, in other embodiments, the forward frame 42 may be configured to support any other suitable ground-engaging tools.

Moreover, like the central and forward frames 40, 42, the aft frame 44 may also be configured to support a plurality of ground-engaging tools. For instance, in the illustrated embodiment, the aft frame is configured to support a plurality of leveling blades 52 and rolling (or crumbler) basket assemblies 54. However, in other embodiments, any other suitable ground-engaging tools may be coupled to and supported by the aft frame 44, such as a plurality of closing disks.

Furthermore, the agricultural implement 12 may also include any number of suitable ground-engaging tool actuators (e.g., hydraulic cylinders) for adjusting the relative positioning of, the penetration depth of, and/or the force being applied to the various ground-engaging tools 46, 50, 52, 54. For instance, in the illustrated embodiment, the implement 12 may include one or more actuators 56 coupled to the central frame 40 for raising and/or lowering the central frame 40 relative to the ground, thereby allowing the penetration depth of and/or the force being applied to the shanks 46 to be adjusted. Similarly, the implement 12 may include one or more actuators 58 coupled to the forward frame 42 to adjust the penetration depth of and/or the force being applied to the disk blades 50.

Additionally, as shown in FIGS. 1 and 2, the agricultural machine 10 may include one or more transceiver-based sensors 102 coupled thereto and/or supported thereon. In general, the transceiver-based sensor(s) 102 is configured to generate surface profile data indicative of the surface profile of a portion of a field across which the agricultural machine 10 is traveling. Specifically, in several embodiments, the transceiver-based sensor(s) 102 may be provided in operative association with the work vehicle 11 such that the transceiver-based sensor(s) 102 has a field(s) of view 104 directed towards a portion(s) of the field disposed in front of or behind the work vehicle 11. Additionally, or alternatively, the transceiver-based sensor(s) 102 may be provided in operative association with the agricultural implement 12 such that the transceiver-based sensor(s) has a field(s) of view 104 directed towards a portion(s) of the field disposed in front of the agricultural implement 12 as the agricultural implement 12 is being towed across the field. In this respect, the transceiver-based sensor(s) 102 may generate surface profile data indicative of the surface profile of one or more portion(s) of the field in front of the agricultural implement 12 (and, more specifically, the shanks 46 of the agricultural implement 12) as the agricultural machine 10 travels across the field. As will be described below, the surface profile data generated by the transceiver-based sensor(s) 102 is used in determining the depth(s) of the compaction layer(s) within the field.

The transceiver-based sensor(s) 102 may correspond to any suitable device(s) configured to generate data indicative of the surface profile of the field. For example, in several embodiments, the transceiver-based sensor(s) 102 may be configured as a LiDAR sensor(s) configured to emit light-based output signals for reflection off of the surface of a portion of the field present within the field(s) of view of such sensor(s) as the agricultural machine 10 travels across the field. Moreover, the LiDAR sensor(s) is configured to detect the reflections of the light-based output signals off of the field surface as return signals. Such return signals, in turn, are indicative of the surface profile of the portion(s) of the field with the field(s) of view of the LiDAR sensor(s). However, in alternative embodiments, the transceiver-based sensor(s) 102 may be configured as any other suitable device(s) for detecting reflection of an emitted signal off of the field surface, such as a radar sensor(s), a time-of-flight camera(s), a scanning ultrasonic sensor(s), etc.

The agricultural machine 10 may include any number of transceiver-based sensor(s) 102 provided at any suitable location(s) that allows surface profile data to be generated as the agricultural machine 10 traverses the field. In this respect, FIGS. 1 and 2 illustrate example locations for mounting the transceiver-based sensor(s) 102 for generating surface profile data for the portion of the field forward of the agricultural implement 12 relative to the direction of travel 34.

For example, as shown in FIG. 1, in one embodiment, one or more transceiver-based sensors 102A may be coupled to the front of the work vehicle 11 such that the transceiver-based sensor(s) 102A has a field(s) of view 104A that allows for the generation of data indicative of the surface profile of an adjacent area or portion of the field disposed in front of the work vehicle 11 and, thus, in front of the agricultural implement 12. In addition to the transceiver-based sensor(s) 102A (or as an alternative thereto), one or more transceiver-based sensors 102B may be coupled to the rear of the work vehicle 11 such that the transceiver-based sensor(s) 102B has a field(s) of view 104B that allows for the generation of data indicative of the surface profile of an adjacent area or portion of the field disposed aft of the work vehicle 11 and forward of the agricultural implement 12. Moreover, in addition to the transceiver-based sensor(s) 102A, 102B (or as an alternative thereto), one or more transceiver-based sensors 102C may also be coupled to the front of the agricultural implement 12 such that the transceiver-based sensor(s) 102C has a field(s) of view 104C that allows for the generation of data indicative of the surface profile of an adjacent area or portion of the field disposed in front of the agricultural implement 12.

However, in alternative embodiments, the transceiver-based sensor(s) 102 may be installed at any other suitable location(s) that allows the device(s) to generate data indicative of the surface profile of a portion(s) of the field forward of the agricultural implement 12 (e.g., forward of the shanks 46 of the agricultural implement 12).

The configuration of the agricultural machine 10 described above and shown in FIGS. 1 and 2 is provided only to place the present subject matter in an exemplary field of use. Thus, the present subject matter may be readily adaptable to any manner of vehicle and/or implement configurations.

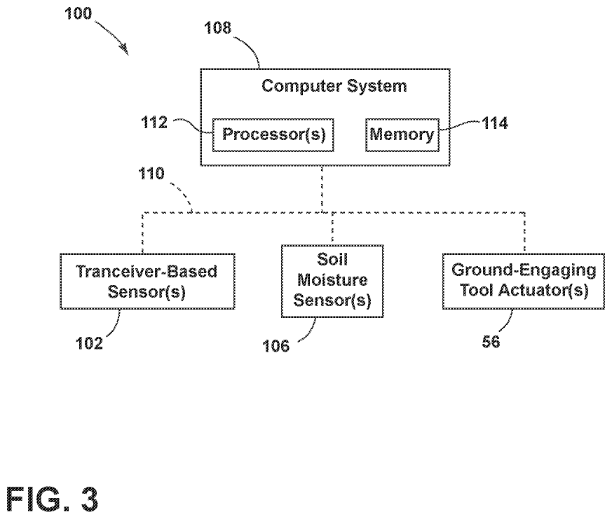

Referring now to FIG. 3, a schematic view of one embodiment of a system 100 for determining compaction layer depth during agricultural machine operation is illustrated in accordance with aspects of the present subject matter. In general, the system 100 will be described herein with reference to the agricultural machine 10 described above with reference to FIGS. 1 and 2. However, it should be appreciated by those of ordinary skill in the art that the disclosed system 100 may generally be utilized with agricultural machines having any other suitable machine configuration.

As shown in FIG. 3, the system 100 includes one or more components of the agricultural machine 10. For example, in the illustrated embodiment, the system 100 includes the transceiver-based sensor(s) 102 and the ground-engaging tool actuator(s) 56. However, in alternative embodiments, the system 100 may include any other suitable components of the agricultural machine 10.

Additionally, the system 100 includes one or more soil moisture sensors 106 coupled to or otherwise supported on the agricultural machine 10. In general, the soil moisture sensor(s) 106 is configured to generate soil moisture data indicative of the soil moisture content of a portion(s) of the field across which the agricultural machine 10 is traveling. As will be described below, the soil moisture data generated by the soil moisture sensor(s) 106 may be used in determining the depth(s) of the compaction layer(s) within the field.

The soil moisture sensor(s) 106 may correspond to any suitable device(s) configured to generate data indicative of the soil moisture content of the field. For example, in several embodiments, the soil moisture sensor(s) 106 may be configured as a microwave-based sensor(s) (e.g., a ground-penetrating radar (GPR) sensor(s)) configured to emit one or more microwave-based output signals directed toward the soil within its field(s) of view. A portion of the microwave-based output signal(s) is, in turn, backscattered or otherwise reflected by the soil as an echo signal(s). In this respect, the soil moisture sensor(s) 106 receives the echo signal(s), which is indicative of a backscattering of the output signal(s) by the soil. Thus, the received echo signal(s) is indicative of the soil moisture content of the portion of the field. However, in alternative embodiments, the soil moisture sensor(s) 106 may be configured as any other suitable device(s) for sensing or detecting the soil moisture content of the field, such as an optical sensor(s), an electromagnetic inductance (EMI) sensor(s), etc.

Moreover, the system 100 includes a computing system 108 communicatively coupled to one or more components of the agricultural machine 10 and/or the system 100 to allow the operation of such components to be electronically or automatically controlled by the computing system 108. For instance, the computing system 108 may be communicatively coupled to the transceiver-based sensor(s) 102 and/or the soil moisture sensor(s) 106 via a communicative link 110. As such, the computing system 108 may be configured to receive data from the transceiver-based sensor(s) 102 and/or the soil moisture sensor(s) 106 that are used to determine the depth(s) of the compaction layer(s) within the field across which the agricultural machine 10 is traveling. Furthermore, the computing system 108 may be communicatively coupled to the ground-engaging tool actuator(s) 56 via the communicative link 110. In this respect, the computing system 108 may be configured to control the operation of the ground-engaging tool actuator(s) 56 to adjust the penetration depths of the shanks 46 of the implement 12 based on the determined compaction layer depth. In addition, the computing system 108 may be communicatively coupled to any other suitable components of the agricultural machine 10 and/or the system 100.

In general, the computing system 108 may include one or more processor-based devices, such as a given controller or computing device or any suitable combination of controllers or computing devices. Thus, in several embodiments, the computing system 108 may include one or more processor(s) 112 and associated memory device(s) 114 configured to perform a variety of computer-implemented functions. As used herein, the term “processor” refers not only to integrated circuits referred to in the art as being included in a computer, but also refers to a controller, a microcontroller, a microcomputer, a programmable logic circuit (PLC), an application specific integrated circuit, and other programmable circuits. Additionally, the memory device(s) 114 of the computing system 108 may generally comprise memory element(s) including, but not limited to, a computer readable medium (e.g., random access memory RAM)), a computer readable non-volatile medium (e.g., a flash memory), a floppy disk, a compact disk-read only memory (CD-ROM), a magneto-optical disk (MOD), a digital versatile disk (DVD) and/or other suitable memory elements. Such memory device(s) 114 may generally be configured to store suitable computer-readable instructions that, when implemented by the processor(s) 112, configure the computing system 108 to perform various computer-implemented functions, such as one or more aspects of the methods and algorithms that will be described herein. In addition, the computing system 108 may also include various other suitable components, such as a communications circuit or module, one or more input/output channels, a data/control bus and/or the like.

The various functions of the computing system 108 may be performed by a single processor-based device or may be distributed across any number of processor-based devices, in which instance such devices may be considered to form part of the computing system 108. For instance, the functions of the computing system 108 may be distributed across multiple application-specific controllers or computing devices, such as a navigation controller, an engine controller, a transmission controller, an implement controller, and/or the like.

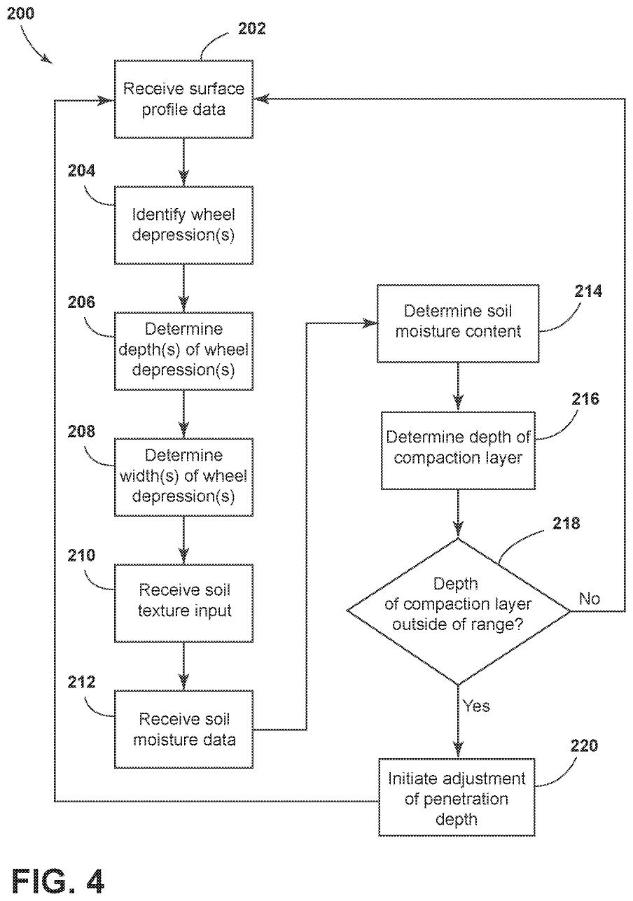

Referring now to FIG. 4, a flow diagram of one embodiment of example control logic 200 that may be executed by the computing system 108 (or any other suitable computing system) for determining compaction layer depth during agricultural machine operation is illustrated in accordance with aspects of the present subject matter. Specifically, the control logic 200 shown in FIG. 4 is representative of steps of one embodiment of an algorithm that can be executed to accurately determine compaction layer depth during agricultural machine operation even as the weight of a given piece of equipment creating the compaction layer varies without requiring substantial computing resources and/or processing time. However, in other embodiments, the control logic 200 may be used in association with any other suitable system, application, and/or the like for determining compaction layer depth during agricultural machine operation.

As shown, at (202), the control logic 200 includes receiving surface profile data indicative of the surface profile of a portion of a field across which an agricultural machine is traveling. Specifically, as mentioned above, in several embodiments, the computing system 108 may be communicatively coupled to the transceiver-based sensor(s) 102 via the communicative link 110. In this respect, as the agricultural machine 10 travels across the field to perform an operation thereon (e.g., a tillage operation), the computing system 108 may receive surface profile data from the transceiver-based sensor(s) 102. Such data may, in turn, be indicative of the surface profile of the portion(s) of the field with the field(s) of view 104 of the transceiver-based sensor(s) 102.

Furthermore, at (204), the control logic 200 includes identifying, with the computing system, a wheel depression within the portion of the field based on the received surface profile data. Specifically, in several embodiments, the computing system 108 is configured to analyze the surface profile data received at (202) to identify any wheel depressions or wheel tracks present within the portion(s) of the field with the field(s) of view 104 of the transceiver-based sensor(s) 102. A wheel depression, in turn, is any cavity, track, rut, divot, depression, low spot, or other abnormality in the surface of profile a field that is formed by a wheel or a track assembly of a vehicle, implement, or other piece of equipment that has traversed the field. For example, in some embodiments, the computing system 108 may identify the wheel depression(s) present within the portion(s) of the field based on shape, such as by identifying rectangular depressions within the surface profile(s). As such, the computing system 108 may include any suitable algorithm(s) stored within its memory device(s) 114 that allows for the identification of wheel depressions within a surface profile generated by a transceiver-based sensor(s). However, in alternative embodiments, the computing system 108 may identify wheel depressions in any other suitable manner.

Additionally, at (206), the control logic 200 includes determining, with the computing system, the depth of the identified wheel depression. Specifically, in several embodiments, the computing system 108 is configured to analyze the surface profile data received at (202) to determine the depth(s) (in the vertical direction) of any wheel depressions identified at (204). As such, the computing system 108 may include any suitable algorithm(s) stored within its memory device(s) 114 that allows for the determination of the depth(s) of the identified wheel depression(s). As will be described below, the computing system 108 is configured to determine the depth(s) of the compaction layer(s) beneath the identified wheel depression(s) based on the depth(s) of the wheel depression(s).

Moreover, at (208), the control logic 200 includes determining, with the computing system, the width of the identified wheel depression. Specifically, in several embodiments, the computing system 108 is configured to analyze the surface profile data received at (202) to determine the width(s) (in the horizontal direction) of any wheel depressions identified at (204). As such, the computing system 108 may include any suitable algorithm(s) stored within its memory device(s) 114 that allows for the determination of the width(s) of the identified wheel depression(s). As will be described below, the computing system 108 may use the width(s) of the wheel depression(s) in controlling the operation of the agricultural machine 10. In some embodiments, (208) may be omitted, such as when depth and width are simultaneously determined.

Moreover, at (210), the control logic 200 includes receiving an input indicative of the texture of soil present within the portion of the field. Specifically, in several embodiments, the computing system 108 is configured to receive one or more inputs indicative of the texture of soil present within the portion(s) of the field. The texture of the soil, in turn, refers to the proportion of sand, silt, and clay particles that make up the mineral fraction of the soil. Thus, the input received at (210) may be any suitable parameter indicative of soil texture, such as the type of soil. Furthermore, at (210), the computing system 108 may receive the input(s) in any suitable manner, such as using received GNSS sensor data and a soil map stored within its memory device(s) 114, from sensor data, from an operator input, etc. As will be described below, the computing system may determine the depth(s) of the compaction layer(s) beneath the identified wheel depression(s) based on the texture of the soil in addition to the determined depth(s) of the identified wheel depression(s). In some embodiments, (210) may be omitted.

In addition, at (212), the control logic 200 includes receiving soil moisture data indicative of the soil moisture content of the portion of the field. Specifically, as mentioned above, in several embodiments, the computing system 108 may be communicatively coupled to the soil moisture sensor(s) 106 via the communicative link 110. In this respect, as the agricultural machine 10 travels across the field to perform the operation thereon, the computing system 108 may receive soil moisture data from the soil moisture sensor(s) 106. Such soil moisture data may, in turn, be indicative of the soil moisture content of the portion of the field.

As shown in FIG. 4, at (214), the control logic 200 includes determining a soil moisture value for the portion of the field based on the received soil moisture sensor data. Specifically, in several embodiments, the computing system 108 is configured to analyze the soil moisture sensor data received at (212) to determine a soil moisture value for the portion of the field. For example, the computing system 108 may include any suitable look-up table(s), mathematical equation(s), and/or algorithm(s) stored within its memory device(s) 114 that correlate the received soil moisture data to the corresponding soil moisture content value(s). As will be described below, the computing system 108 may determine the depth(s) of the compaction layer beneath the identified wheel depression(s) based on the determined soil moisture content and the determined depth(s) of the identified wheel depression(s). In some embodiments, (212) and (214) may be omitted.

Furthermore, at (216), the control logic 200 includes determining the depth of the compaction layer beneath the identified wheel depression based on the determined depth of the identified wheel depression. Specifically, in several embodiments, the computing system 108 is configured to determine the depth(s) of the compaction layer(s) beneath the wheel depression(s) identified at (204) based on the depth(s) of the wheel depression(s) determined at (206). For example, the computing system 108 may include any suitable look-up table(s), mathematical equation(s), and/or algorithm(s) stored within its memory device(s) 114 that correlate the depth(s) of the wheel depression(s) to the corresponding depth(s) of the compaction layer(s) beneath the wheel depression(s). Additionally, in some embodiments, the width(s) of the wheel depression(s) determined at (208) may be used in addition to the determined depth(s) of the wheel depression(s) to determine the depth(s) of the compaction layer(s). In such embodiments, the combination of the width(s) and depth(s) may provide an indication of the pressure exerted by the wheel(s) forming the wheel depression(s). Thus, the control logic 200 provides accurate indications of compaction layer depth beneath wheel depressions in a field even as the weight of a given piece of equipment creating such compaction layers varies.

Additionally, at (216), in some embodiments, other parameters may be used in addition to the depth(s) of the wheel depression(s) determined at (206) to determine compaction layer depth. For example, in one embodiment, the texture of the soil within the field may be used to the depth(s) of the wheel depression(s) as texture can provide an indication of how much the soil compresses under a wheel/track. Thus, in such an embodiment, the computing system 108 may be configured to determine the depth(s) of the compaction layer(s) beneath the identified wheel depression(s) based on the texture of the soil (e.g., as determined from the input(s) received at (210)) and the depth(s) of the wheel depression(s) determined at (206). Alternatively, or additionally, the soil moisture content of the field may be used as soil moisture can provide an indication of how much the soil compresses. Thus, in such an embodiment, the computing system 108 may be configured to determine the depth(s) of the compaction layer(s) beneath the identified wheel depression(s) based on the soil moisture content determined at (214) and the depth(s) of the wheel depression(s) determined at (206).

As will be described below, the computing system 108 may be configured to control one or more operating parameters of the agricultural machine 10 (e.g., the penetration depths of its ground-engaging tools, such as the shanks 46) based on the determined depth(s) of the compaction layer(s). Additionally, in some embodiments, the computing system 108 may be configured to control the operating parameter(s) of the agricultural machine 10 based on the width(s) of the wheel depression(s) determined at (208) in addition to the depth(s) of the compaction layer(s) determined at (206).

Moreover, at (218), the control logic 200 includes comparing the determined depth of the compaction layer to a predetermined range. Specifically, in several embodiments, the computing system 108 is configured to compare the depth(s) of the compaction layer(s) determined at (216) to a predetermined range. When the determined depth(s) of the compaction layer(s) is within the predetermined range, the shanks or other ground-engaging tools of the agricultural machine 10 are properly positioned to break up the identified compaction layer(s). In such instances, the control logic 200 returns to (202). Conversely, when the determined depth(s) of the compaction layer(s) is outside of the predetermined range, the shanks or other ground-engaging tools of the agricultural machine 10 are not properly positioned to break up the identified compaction layer(s). In such instances, the control logic 200 proceeds to (220).

In addition, at (220), the control logic 200 includes initiating an adjustment to the penetration depth of the ground-engaging tool when the determined depth of the compaction layer falls outside of the predetermined range. Specifically, in several embodiments, when it is determined at (218) that the determined depth(s) of the compaction layer(s) is outside of the predetermined range, the computing system 108 is configured to initiate an adjustment to the penetration depth of the ground-engaging tools of the agricultural machine 10, such as the shanks 46. For example, in one embodiment, the computing system 108 may be configured to transmit control signals to the ground-engaging tool actuator(s) 56 via the communicative link 110. Such control signals, in turn, instruct the ground-engaging tool actuator(s) 56 to adjust the penetration depth of the shanks 46.

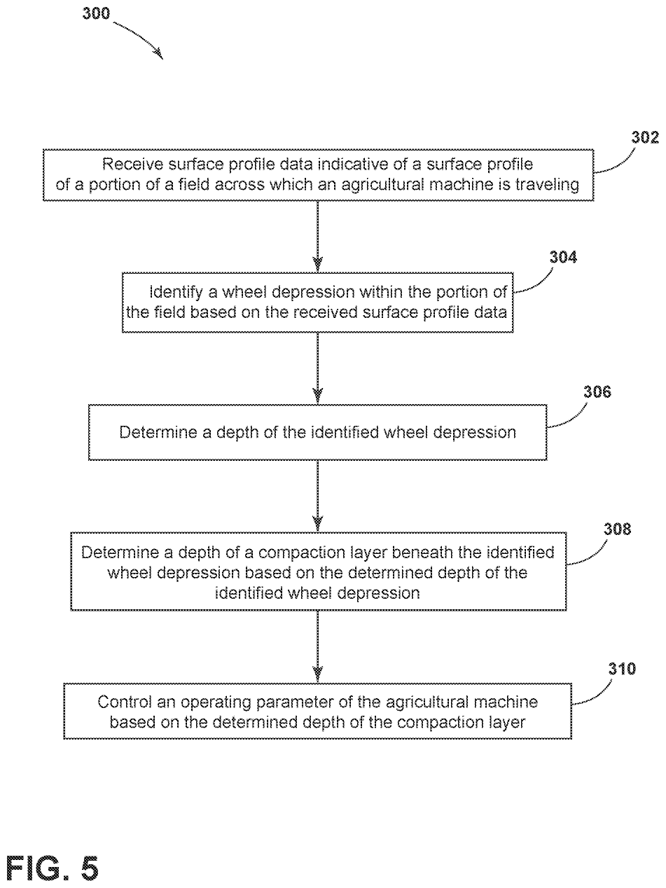

Referring now to FIG. 5, a flow diagram of one embodiment of a method 300 for determining compaction layer depth during agricultural machine operation is illustrated in accordance with aspects of the present subject matter. In general, the method 300 will be described herein with reference to the agricultural machine 10 and the system 100 described above with reference to FIGS. 1-4. However, it should be appreciated by those of ordinary skill in the art that the disclosed method 300 may generally be implemented with any agricultural machine having any suitable machine configuration and/or within any system having any suitable system configuration. In addition, although FIG. 5 depicts steps performed in a particular order for purposes of illustration and discussion, the methods discussed herein are not limited to any particular order or arrangement. One skilled in the art, using the disclosures provided herein, will appreciate that various steps of the methods disclosed herein can be omitted, rearranged, combined, and/or adapted in various ways without deviating from the scope of the present disclosure.

As shown in FIG. 5, at (302), the method 300 includes receiving, with a computing system, surface profile data indicative of a surface profile of a portion of a field across which an agricultural machine is traveling. For instance, as described above, the computing system 108 may be configured to receive surface profile data from the transceiver-based sensor(s) 102. The surface profile data, in turn, is indicative of the surface profile of a portion(s) of the field across which the agricultural machine 10 is traveling.

Furthermore, at (304), the method 300 includes identifying, with the computing system, a wheel depression within the portion of the field based on the received surface profile data. For instance, as described above, the computing system 108 may be configured to identify one or more wheel depressions within the portion(s) of the field based on the received surface profile data.

Additionally, at (306), the method 300 includes determining, with the computing system, a depth of the identified wheel depression. For instance, as described above, the computing system 108 may be configured to determine the depth(s) of the identified wheel depression(s).

Moreover, at (308), the method 300 includes determining, with the computing system, a depth of a compaction layer beneath the identified wheel depression based on the determined depth of the identified wheel depression. For instance, as described above, the computing system 108 may be configured to determine the depth(s) of the compaction layer(s) beneath the identified wheel depression(s) based on the determined depth(s) of the identified wheel depression(s).

In addition, at (310), the method 300 includes controlling, with the computing system, an operating parameter of the agricultural machine based on the determined depth of the compaction layer. For instance, as described above, the computing system 108 may be configured to control one or more operating parameters of the agricultural machine 10 based on the determined depth(s) of the compaction layer(s). For example, in one embodiment, the computing system 108 may control the operation of the ground-engaging tool actuator(s) 56 to adjust the penetration depth of the shanks 46 based on the determined depth(s) of the compaction layer(s).

It is to be understood that the steps of the control logic 200 and the method 300 are performed by the computing system 108 upon loading and executing software code or instructions which are tangibly stored on a tangible computer readable medium, such as on a magnetic medium, e.g., a computer hard drive, an optical medium, e.g., an optical disc, solid-state memory, e.g., flash memory, or other storage media known in the art. Thus, any of the functionality performed by the computing system 108 described herein, such as the control logic 200 and the method 300, is implemented in software code or instructions which are tangibly stored on a tangible computer readable medium. The computing system 108 loads the software code or instructions via a direct interface with the computer readable medium or via a wired and/or wireless network. Upon loading and executing such software code or instructions by the computing system 108, the computing system 108 may perform any of the functionality of the computing system 108 described herein, including any steps of the control logic 200 and the method 300 described herein.

The term “software code” or “code” used herein refers to any instructions or set of instructions that influence the operation of a computer or controller. They may exist in a computer-executable form, such as machine code, which is the set of instructions and data directly executed by a computer's central processing unit or by a controller, a human-understandable form, such as source code, which may be compiled in order to be executed by a computer's central processing unit or by a controller, or an intermediate form, such as object code, which is produced by a compiler. As used herein, the term “software code” or “code” also includes any human-understandable computer instructions or set of instructions, e.g., a script, that may be executed on the fly with the aid of an interpreter executed by a computer's central processing unit or by a controller.

This written description uses examples to disclose the technology, including the best mode, and also to enable any person skilled in the art to practice the technology, including making and using any devices or systems and performing any incorporated methods. The patentable scope of the technology is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal language of the claims.

1. An agricultural machine, comprising:
a frame;
a plurality of wheels coupled to the frame;
a transceiver-based sensor supported on the frame, the transceiver-based sensor configured to generate surface profile data indicative of a surface profile of a portion of a field across which the agricultural machine is traveling; and
a computing system communicatively coupled to the transceiver-based sensor, the computing system configured to:
identify a wheel depression within the portion of the field based on the surface profile data generated by the transceiver-based sensor;
determine a depth of the identified wheel depression;
determine a depth of a compaction layer beneath the identified wheel depression based on the determined depth of the identified wheel depression; and
control an operating parameter of the agricultural machine based on the determined depth of the compaction layer.

2. The agricultural machine of claim 1, wherein the computing system is further configured to:
receive an input indicative of a texture of soil present within the portion of the field; and
determine the depth of the compaction layer beneath the identified wheel depression based on the texture of the soil and the determined depth of the identified wheel depression.

3. The agricultural machine of claim 1, further comprising:
a soil moisture sensor configured to generate soil moisture data indicative of a soil moisture content of the portion of the field,
wherein the computing system is further configured to:
determine the soil moisture content of the portion of the field based on the soil moisture data generated by the soil moisture sensor; and
determine the depth of the compaction layer beneath the identified wheel depression based on the determined soil moisture content and the determined depth of the identified wheel depression.

4. The agricultural machine of claim 1, further comprising:
a ground-engaging tool configured to penetrate into soil within the field to a penetration depth,
wherein the operating parameter comprises the penetration depth of the ground-engaging tool.

5. The agricultural machine of claim 1, wherein the transceiver-based sensor has a field of view directed forward of the agricultural machine relative to a direction of travel of the agricultural machine.

6. A system for determining compaction layer depth during agricultural machine operation, the system comprising:
a transceiver-based sensor configured to generate surface profile data indicative of a surface profile of a portion of a field across which an agricultural machine is traveling; and
a computing system communicatively coupled to the transceiver-based sensor, the computing system configured to:
identify a wheel depression within the portion of the field based on the surface profile data generated by the transceiver-based sensor;
determine a depth of the identified wheel depression; and
determine a depth of a compaction layer beneath the identified wheel depression based on the determined depth of the identified wheel depression.

7. The system of claim 6, wherein the computing system is further configured to:
receive an input indicative of a texture of soil present within the portion of the field; and
determine the depth of the compaction layer beneath the identified wheel depression based on the texture of the soil and the determined depth of the identified wheel depression.

8. The system of claim 6, further comprising:
a soil moisture sensor configured to generate soil moisture data indicative of a soil moisture content of the portion of the field,
wherein the computing system is further configured to:
determine the soil moisture content of the portion of the field based on the soil moisture data generated by the soil moisture sensor; and
determine the depth of the compaction layer beneath the identified wheel depression based on the determined soil moisture content and the determined depth of the identified wheel depression.

9. The system of claim 6, wherein, when identifying the wheel depression, the computing system is configured to identify the wheel depression within the portion of the field within the data generated by the transceiver-based sensor based on shape.

10. The system of claim 6, wherein the computing system is further configured to:
control an operating parameter of the agricultural machine based on the determined depth of the compaction layer.

11. The system of claim 10, wherein the computing system is further configured to:
determine a width of the identified wheel depression; and
control the operating parameter of the agricultural machine based on the determined width of the identified wheel depression and the determined depth of the compaction layer.

12. The system of claim 11, further comprising:
a ground-engaging tool configured to penetrate into soil within the field to a penetration depth,
wherein the operating parameter comprises the penetration depth of the ground-engaging tool.

13. The system of claim 12, wherein, when controlling the operating parameter, the computing system is configured to:
compare the determined depth of the compaction layer to a predetermined range; and
initiate an adjustment to the penetration depth of the ground-engaging tool when determined depth of the compaction layer falls outside of the predetermined range.

14. The system of claim 12, wherein the ground-engaging tool comprises a shank.

15. The system of claim 6, wherein the transceiver-based sensor comprises a LiDAR sensor.

16. A method for determining compaction layer depth during agricultural machine operation, the method comprising:
receiving, with a computing system, surface profile data indicative of a surface profile of a portion of a field across which an agricultural machine is traveling;
identifying, with the computing system, a wheel depression within the portion of the field based on the received surface profile data;
determining, with the computing system, a depth of the identified wheel depression;
determining, with the computing system, a depth of a compaction layer beneath the identified wheel depression based on the determined depth of the identified wheel depression; and
controlling, with the computing system, an operating parameter of the agricultural machine based on the determined depth of the compaction layer.

17. The method of claim 16, further comprising:
receiving, with the computing system, an input indicative of a texture of soil present within the portion of the field; and
determining, with the computing system, the depth of the compaction layer beneath the identified wheel depression based on the texture of the soil and the determined depth of the identified wheel depression.

18. The method of claim 16, further comprising:
receiving, with the computing system, soil moisture data indicative of a soil moisture content of the portion of the field,
determining, with the computing system, the soil moisture content of the portion of the field based on the received soil moisture data; and
determining, with the computing system, the depth of the compaction layer beneath the identified wheel depression based on the determined soil moisture content and the determined depth of the identified wheel depression.

19. The method of claim 16, wherein identifying the wheel depression comprises identifying, with the computing system, the wheel depression within the portion of the field within the soil profile data based on shape.

20. The method of claim 19, further comprising:
determining, with the computing system, a width of the identified wheel depression; and
controlling, with the computing system, the operating parameter of the agricultural machine based on the determined width of the identified wheel depression and the determined depth of the compaction layer.
